(19) United States
(12) Patent Application Publication
(10) Pub. No.: US 2024/0024354 A1
(43) Pub. Date: Jan. 25, 2024

(54) MATERIALS AND METHODS OF TREATING VIRAL INFECTION WITH AMPHIPHILIC BLOCK COPOLYMERS

(71) Applicant: The University of Chicago, Chicago, IL (US)

(72) Inventors: Raphael C. LEE, Chicago, IL (US); Michelle X. LING, Chicago, IL (US); Michelle NGUYEN, Conyers, GA (US); Kyle J. MCCOLLUM, Tucson, AZ (US); Victoria A. BIGDELLE, Chicago, IL (US)

(21) Appl. No.: 18/024,700

(22) PCT Filed: Sep. 7, 2021
(86) PCT No.: PCT/US2021/049266

§ 371 Date: Mar. 3, 2023

Related U.S. Application Data
(60) Provisional application No. 63/074,951, filed on Sep. 4, 2020.

(51) Int. Cl. A61K 31/765; A61P 31/14

(52) U.S. Cl. CPC A61K 31/765; A61P 31/14; A61K 2121/00

(57) ABSTRACT

Provided is a method of contacting a subject, cell, or tissue with an amphiphilic block copolymer to treat a viral infection. The resulting effect can be treating a disease caused by a virus, inhibiting viral replication, inhibiting an unfolded protein response of a virus, preventing death of tissue infected by a virus, or promoting cell repair and recovery to increase survival of cells infected by a virus. The hydrophobic block of the amphiphilic block copolymer binds to an exposed hydrophobic domain of the virus. Further provided is an amphiphilic block copolymer comprising three or more hydrophobic substituents or an alkylene spacer on a hydrophobic block of the copolymer.

### CROSS-REFERENCE TO A RELATED APPLICATION

This patent application is the U.S. national stage application corresponding to PCT patent application PCT/US2021/049266 with the international filing date of Sep. 7, 2021 and claiming the benefit of priority to U.S. Provisional Patent Application No. 63/074,951, filed Sep. 4, 2020, the entire disclosure of both applications is herein incorporated by reference in their entirety.

### BACKGROUND

There continues to be a need for inhibition of viral replication and treatment of viral infections including but not limited to SARS-CoV-2, the virus that has caused the COVID-19 global pandemic of 2020.

### SUMMARY

In an embodiment, the invention provides a method of treating a disease or infection caused by a virus in a subject in need thereof comprising administering to the subject a therapeutically effective amount of an amphiphilic block copolymer or an amphiphilic polymer-polypeptide conjugate.

In an embodiment, the invention also provides a method of inhibiting virus replication in a subject infected by a virus comprising administering to the subject a therapeutically effective amount of an amphiphilic block copolymer.

In an embodiment, the invention further provides a method of inhibiting virus replication in cells or tissues infected by a virus comprising an exposed hydrophobic domain comprising contacting the exposed hydrophobic domain with an amphiphilic block copolymer.

In an embodiment, the invention further provides a method of inhibiting a gene transcription response to a viral infection comprising an exposed hydrophobic domain comprising contacting the exposed hydrophobic domain with an amphiphilic block copolymer.

In an embodiment, the invention further provides a method of inhibiting a cellular metabolic response to a viral infection comprising an exposed hydrophobic domain comprising contacting the exposed hydrophobic domain with an amphiphilic block copolymer.

In an embodiment, the invention further provides a method of inhibiting an unfolded protein response of a virus comprising an exposed hydrophobic domain comprising contacting the exposed hydrophobic domain with an amphiphilic block copolymer.

In an embodiment, the invention provides a method of preventing growth of tissue infected by a virus comprising contacting the infected tissue with an amphiphilic block copolymer.

In an embodiment, the invention provides a method of preventing death of tissue infected by a virus comprising contacting the infected tissue with an amphiphilic block copolymer.

In an embodiment, the invention additionally provides a method of promoting cell repair and recovery to increase survival of cells infected by a virus comprising contacting the infected cells with an amphiphilic block copolymer.

In an embodiment, the invention provides an amphiphilic block copolymer comprising three or more hydrophobic substituents or an alkylene spacer on a hydrophobic block of the copolymer.

Additional embodiments are as described herein.

### BRIEF DESCRIPTION OF THE DRAWINGS

FIG. 1 is a schematic illustrating possible sites of the binding of an amphiphilic block copolymer to a virus either by blocking entry into a cell or by blocking an unfolded protein response (UPR) in accordance with embodiments of the invention.

FIG. 2 is a graph illustrating the percent of human lung cells infected with coronavirus OC43 after treatment of amphiphilic poloxamers and poloxamine of varying molecular weights relative to a no drug control in accordance with embodiments of the invention.

FIG. 3 are dose response curves of drug concentration (mM) versus percent of human lung cells infected with SARS-CoV-2 relative to a no drug control using amphiphilic poloxamers and poloxamine of varying molecular weights in accordance with embodiments of the invention.

FIG. 4 is a dose response curve of drug concentration (mM) versus percent of cells infected with SARS-CoV-2 virus relative to a no drug control using P118 alone (FIG. 4A) and P108 in combination with ascorbate (“VC”) as an antioxidant (FIG. 4B) in accordance with embodiments of the invention.

FIG. 5 is a dose response curve of drug concentration (mM) versus percent of cells infected with SARS-CoV-2 virus relative to a no drug control using P188 alone (FIG. 5A) and P188 in combination with VC as an antioxidant (FIG. 5B) in accordance with embodiments of the invention.

FIG. 6 is a dose response curve of drug concentration (mM) versus percent of cells infected with SARS-CoV-2 virus relative to a no drug control using P238 alone (FIG. 6A) and P238 in combination with VC as an antioxidant (FIG. 6B) in accordance with embodiments of the invention.

FIG. 7 is a dose response curve of drug concentration (mM) versus percent of cells infected with SARS-CoV-2 virus relative to a no drug control using T1107 alone (FIG. 7A) and T1107 in combination with VC as an antioxidant (FIG. 7B) in accordance with embodiments of the invention.

FIG. 8 is a dose response curve of drug concentration (mM) versus percent of cells infected with SARS-CoV-2 virus relative to a no drug control using VC and no amphiphilic block copolymer in accordance with embodiments of the invention.

FIG. 9 is a series of images of the predicted interaction between poloxamer and the SARS-CoV-2 Spike Protein obtained by computational molecular dynamic modeling.

FIG. 9A depicts the lack of interaction between Poloxamer 108 and the Spike Protein. FIG. 9B depicts the binding of Poloxamer 188 to the S1 subunit of the Spike Protein. The S1 subunit is the section of the Spike Protein that contains the ACE II binding site which mediates the SARS-CoV-2 entry into mammalian cells.

### DETAILED DESCRIPTION

In an embodiment, the present invention provides a method of treating a disease caused by a virus in a subject in need thereof comprising administering to the subject a therapeutically effective amount of an amphiphilic block copolymer.

In an embodiment, the invention also provides a method of inhibiting virus replication in a subject infected by a virus comprising administering to the subject a therapeutically effective amount of an amphiphilic block copolymer.

In an embodiment, the invention further provides a method of inhibiting a gene transcription response to a viral infection comprising an exposed hydrophobic domain comprising contacting the exposed hydrophobic domain with an amphiphilic block copolymer.

In an embodiment, the invention further provides a method of inhibiting a cellular metabolic response to a viral infection comprising an exposed hydrophobic domain comprising contacting the exposed hydrophobic domain with an amphiphilic block copolymer.

In an embodiment, the invention further provides a method of inhibiting an unfolded protein response (UPR) of a virus comprising an exposed hydrophobic domain comprising contacting the exposed hydrophobic domain with an amphiphilic block copolymer.

In an embodiment, the invention provides a method of preventing death of tissue infected by a virus comprising contacting the infected tissue with an amphiphilic block copolymer.

In an embodiment, the invention additionally provides a method of preventing cell protection to lessen cellular stress responses and increase survival of cells infected by a virus comprising contacting the infected cells with an amphiphilic block copolymer.

When a virus enters or fuses with a host cell, the virus takes control of the cell's protein synthesis capability, which includes the endoplasmic reticulum (ER), to mass produce viral proteins. This results in an accumulation of viral proteins in the cytoplasm of the cell. Since these viral proteins have exposed hydrophobic domains, it reduces cytoplasmic water activity. To compensate for this, cells have to consume larger amounts of adenosine triphosphate (ATP), which would lead to oxidative stress which activate a production of stress proteins as well as either cell proliferation or cell death responses.

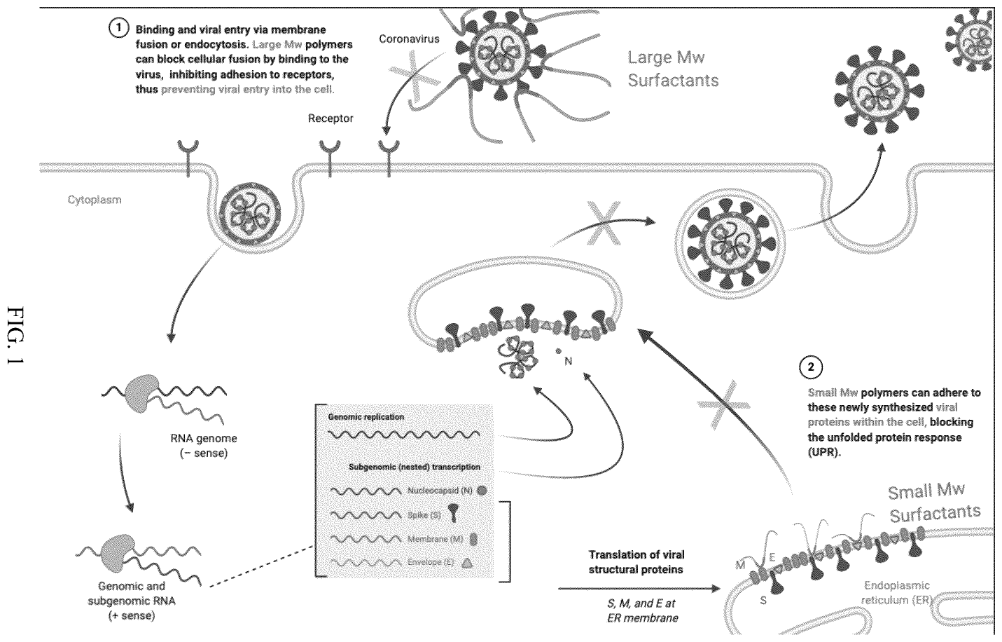

In addition, intracellular proteins with exposed hydrophobic domains activate cellular detection mechanisms that activate genes that encode cellular stress proteins called the Unfolded Protein Response. Without wishing to be bound by any theory, an amphiphilic block copolymer (e.g., a large molecular weight amphiphilic block copolymer) can inhibit viral entry into the cell by binding to the virus surface proteins, which inhibits adhesion to cell surface proteins or the cell bilayer lipid membrane. See FIG. 1. Preventing the virus from entering cells and thereby inhibiting its replication leads to a therapeutic method of treating a disease caused by a virus.

In a particular example, molecular modeling suggests that the hydrophobic block of the amphiphilic block copolymer interacts with the hydrophobic heptad repeat 2 (HR2) domain found in SARS-CoV-2, SARS-CoV, and MERS-CoV viruses, which plays a role in allowing the virus to enter a cell. Without wishing to be bound by any theory, the alpha helical HR2 domain facilitates the formation of a fusion pore in the cellular membrane which then allows the virus to infect the cell. Computational molecular dynamic modeling suggests that the hydrophobic block of the amphiphilic block copolymer (e.g., an amphiphilic block copolymer modified to have three or more hydrophobic substituents or an alkylene spacer on a hydrophobic block) can interact with the spike protein in such a way that the copolymer prevents entry of the virus into the cell and cell-cell fusion. In a particular example, commercially available Poloxamer 188 modified with a propylene spacer in the middle of the polyoxypropylene hydrophobic block provides more opportunities for hydrophobic interactions through hydrogen bonding, thereby stabilizing polymer-virus interactions. The molecular model shows the polymer interacting with the exposed hydrophobic region of the HR2 domain with a 10 ns simulation.

Alternatively, without wishing to be bound by any theory, once a virus has entered a cell, an amphiphilic block copolymer (e.g., a small molecular weight amphiphilic block copolymer) can adhere to newly synthesized viral proteins within the cell, thereby blocking the Unfolded Protein Response (UPR) or reduce further production of viral proteins. See FIG. 1. Blocking the UPR leads to reducing the cellular stress response, thus increasing survival of cells and tissues that have been infected by a virus and/or preventing cell protection to increase survival of cells (i.e., cell viability) infected by the virus and reduce inflammation.

The methods described herein are applicable to any virus with an exposed hydrophobic domain (region), including ribonucleic acid (RNA) viruses with an exposed hydrophobic domain and deoxyribonucleic acid (DNA) viruses with an exposed hydrophobic domain. The term “exposed hydrophobic domain” refers to one or more viral surface proteins that have potential to form hydrophobic bonds.

In some instances, the virus is an RNA virus. The RNA virus can be, for example, a coronavirus (e.g., 229E, NL63, OC43, HKU1, MERS-CoV, SARS-CoV, or SARS-CoV-2), a flavivirus (e.g., a hepacivirus, such as hepatitis C virus (protein E2), Japanese encephalitis virus (NS2A protein), dengue virus, Zika virus (ZIKV)), a rhabdovirus (e.g., vesicular stomatitis virus (M protein)), an orthmyxovirus (e.g., an influenza A virus, such as NS1 and HA), a hepevirus (e.g., hepatitis E virus (ORF2 protein)), a herpesvirus (e.g., Epstein Barr virus (EV71 protein) and cytomegalovirus (DNA virus, US11 and pUL38 protein)), or a retrovirus (e.g., human immunodeficiency virus (HIV)). In some embodiments, the RNA virus is a coronavirus, such as 229E, NL63, OC43, HKU1, MERW-CoV, SARS-CoV, or SARS-CoV-2. Preferably, the coronavirus is SARS-CoV-2.

In some instances, the virus is a DNA virus. The DNA virus can be, for example, a hepadnavirus (e.g., hepatitis B virus (S protein)), an asfarvirus (African swine fever virus), a papillomavirus (e.g., human papillomavirus (F6 protein)), or a poxvirus (e.g., vaccinia virus (E3L protein)).

Amphiphilic block copolymers serve as active agents in the inventive methods. Amphiphilic block copolymers are desirable for multiple reasons as they have low detergency, high biocompatibility, do not denature proteins, do not disrupt cell membranes, and the effectiveness of which are not affected by viral mutations. Additionally, amphiphilic block copolymers are easily synthesized, can be modified to have pendant groups, have highly tunable molecular weights, and many are approved by the Food and Drug Administration (FDA). Without wishing to be bound by theory, it is believed that the hydrophilic blocks are able to disrupt the water structure surrounding the protein and create steric bulk, while the hydrophobic blocks bind to exposed hydrophobic domains to inhibit UPR, viral entry, viral attachment to the cell, and viral replication.

The amphiphilic block copolymer comprises both hydrophilic (polar) (“A”) and hydrophobic (nonpolar) (“B”) regions and acts as a surfactant. The block copolymer structure can be hydrophilic-hydrophobic-hydrophilic (ABA), hydrophobic-hydrophilic-hydrophobic (BAB), or have a core structure (A or B) with two or more pendant side chains of the structure -A (if a B core), -B (if an A core), -AB, -BA, -ABA, -BAB, or a combination thereof. An amphiphilic block copolymer that is biocompatible and/or FDA approved is preferred.

In general, the amphiphilic block copolymer comprises at least one (e.g., 1, 2, 3, 4, 5, etc.) hydrophobic block and at least one (e.g., 1, 2, 3, 4, 5, etc.) hydrophilic block. The hydrophilicity and hydrophobicity can be measured, if necessary, by any suitable method. For example, hydrophilic-lipophilic balance (HLB) of the amphiphilic block copolymer can be measured by Griffin's method, which uses the equation:

HLB=20×(M(hydrophilic)/(M(hydrophobic)+M(hydrophilic)),

in which M is the molecular mass of the hydrophilic and hydrophobic portions of the copolymer. In general, copolymers are considered to be hydrophobic when HLB is between 1-7 (i.e., 1, 2, 3, 4, 5, 6, or 7), and copolymers are considered to be hydrophilic when HLB is greater than 7 (e.g., 8 or more, such as 8, 9, 10, 11, 12, 13, 14, 15, 16, 17, 18, 19, or 20).

In some embodiments, the hydrophobic block comprises repeat units selected from a hydrophobic polypeptide, polyoxypropylene, polystyrene, polyglycolide, polylactide, poly(lactic-glycoacid), polycaprolactone, hydrophobic polyurethane, polyester, poly-N-isopropylacrylamide, polymethylmethacrylate, poly(2-dimethylaminoethylmethacrylate), polyethylene, polypropylene, polyisoprene, polybutylene, polybutadiene, poly(styrene-butadiene), polyvinyl chloride, polytetrafluoroethylene, polydimethylsiloxane, and a combination thereof.

In some embodiments, the hydrophilic block comprises repeat units selected from polyalkylene oxide (e.g., C₁-C₁₀ polyalkylene oxide, such as polymethylene oxide, polyethylene oxide, polypropylene oxide, polybutylene oxide, polypentylene oxide, polyhexylene oxide, polyheptylene oxide, polyoctylene oxide, polynonylene oxide, and polydecylene oxide, including branched and structural isomers thereof), polyvinyl alcohol, hydrophilic polyurethane, polyvinylpyrrolidone, polyacrylamide, polyacrylic acid, poly(meth)acrylic acid, polyethylenimine, poly(methyl vinyl ether), poly(styrene-maleic anhydride), polyethylene glycol ether, polyamine, a hydrophilic polypeptide, and a combination thereof. Preferably the hydrophilic block comprises polyethylene oxide.

Polyurethanes are prepared from polyols and diisocyanates to form repeat units with an —NH—CO—O— linkage. Whether a polyurethane is hydrophilic or hydrophobic typically depends on the monomers used. In general, the hydrophilic to hydrophobic content ratio can be controlled by using a mixture of polyols with varying hydrophilicities and/or the use of chain extenders. The polyols are generally based on polyesters, polyethers, mixtures thereof, and copolymers of esters with ethers. Polyurethanes based on polyethylene oxide are highly hydrophilic materials.

Suitable examples of diisocyanates include 1,6-hexamethylene diisocyanate, 1,4-diisocyanato butane, L-lysine diisocyanate, isophorone diisocyanate, 1,4-diisocyanato 2-methyl butane, 2,3-diisocyanato 2,3-dimethyl butane, 1,4-di(1propoxy-3-diisocyanate, 1,4-diisocyanato 2-butene, 1,10-diisocyanato decane, ethylene diisocyanate, 2,5 bis(2-isocyanato ethyl) furan, 1,6-diisocyanato 2,5-diethyl hexane, 1,6-diisocyanato 3-methoxy hexane, 1,5 diisocyanato pentane, 1,12-dodecamethylene diisocyanate, 2 methyl-2,4 diisocyanato pentane, 2,2 dimethyl-1,5 diisocyanato pentane, ethyl phosphonyl diisocyanate, 2,2,4-trimethyl-1,6-hexamethylene diisocyanate, 4,4′-diphenylmethane diisocyanate, 2,4′-diphenylmethane diisocyanate and 2,2′-diphenylmethane diisocyanate; mixtures of 2,4′-diphenylmethane diisocyanate and 4,4′-diphenylmethane diisocyanate, 2,4-toluene diisocyanate, mixtures of 2,4-toluene diisocyanate and 2,6-toluene diisocyanate, 2,4′-diphenylmethane diisocyanates, 4,4′-1-diphenylethane diisocyanato, 1,5-naphthylene diisocyanate, and combinations thereof.

The chain extenders are low molecular weight diols, diamines, triols, or triamines, or higher molecular weight oligomeric units having the functionality of two or higher. Suitable chain extenders include water, aliphatic difunctional or trifunctional alcohols, amines, aminoalcohols, aminoacids, and hydroxyacids. Specific examples include 2-aminoethanol, 2-dibutylaminoethanol, n-alkyldiethanolamines, n-methyl-diethanolamine, ethylene diol, diethylene diol, 1,4-butanediol, propylene diol, dipropylene diol, 1,6-hexanediol, isosorbide (1,4:3,6-dianhydrosorbitol), glycerol, ethylene diamine, tetramethylene diamine, hexamethylene diamine, isophorone diamine, propanolamine, ethanolamine, glycyl-L-glutamine, glycyl-L-tyrosine, L-glutathione, glycylglycine, L-malic acid, and combinations thereof.

In certain preferred embodiments, the amphiphilic block copolymer comprises repeat units comprising a polypeptide, a poloxamer, a meroxapol, a poloxamine, a polyol, a polyethylenimine, a styrene maleic anhydride, or a combination thereof in di-block, tri-block, tetra-block or more compositions.

In any of the embodiments herein, the amphiphilic block copolymer comprises a polypeptide. A polypeptide can be hydrophilic or hydrophobic depending on the particular amino acids forming the polypeptide. Hydrophobic amino acids include glycine, alanine, valine, leucine, isoleucine, proline, phenylalanine, methionine, and tryptophan. Hydrophilic amino acids have side chains that are polar but not charged, including serine, threonine, cysteine, asparagine, glutamine, and tyrosine. The polypeptide can include any desirable sequence of two or more amino acids to provide the desired hydrophilicity or hydrophobicity.

In any of the embodiments herein, the amphiphilic block copolymer comprises a poloxamer. A poloxamer is a triblock ABA copolymer in which a central hydrophobic polyoxypropylene core connected to two hydrophilic polyethylene oxide side chains. Poloxamers are synthesized by the sequential addition of propylene oxide, followed by ethylene oxide, to propylene glycol, which in the case of the poloxamers constitutes the water-soluble organic component of the polymer. The inner polypropylene oxide is the hydrophobic portion of the poloxamer. This is due to the fact that this group changes from a water-soluble to a water-insoluble polymer as the molecular weight goes above 750 g/mol. Adding ethylene oxide in the final step makes the molecule water-soluble.

In an embodiment, the poloxamer has a structure of formula (I)

HO—(C₂H₄O)_{b}—(C₃H₆O)ₐ—(C₂H₄O)_{b}—H (I)

in which a is an integer such that the hydrophobic polyoxypropylene core (C₃H₆O) has a molecular weight of about 500-15,500 g/mol, and b is an integer such that the hydrophilic polyethylene oxide side chains (C₂H₄O)_{b} constitute about 50-90% by weight of the poloxamer.

Suitable examples of a poloxamer include poloxamer 108 (P108), poloxamer 124 (P124), poloxamer 188 (P188), poloxamer 237 (P237), poloxamer 238 (P238), poloxamer 331 (P331), poloxamer 338 (P338), poloxamer 407 (P407), and a poloxamer ester of fatty acid (PEFA) (e.g., ethylene oxide/propylene oxide copolymer fatty acid ester, polyoxyethylene/polyoxypropylene copolymer fatty acid diester, polyoxyethylene-polyoxypropylene block copolymer fatty acid ester, polyethylene oxide-polypropylene oxide block copolymer fatty acid ester, polyethylene/polypropylene glycol fatty acid ester).

In any of the embodiments herein, the amphiphilic block copolymer comprises a meroxapol. A meroxapol is a triblock BAB copolymer in which a central hydrophilic polyethylene oxide block (core) is connected to two hydrophobic polyoxypropylene side chains. Compared to poloxamers, the order of addition of the alkylene oxides is reversed to produce a meroxapol. Ethylene glycol is the initiator, which provides secondary hydroxyl groups at the termini.

In any of the embodiments herein, the amphiphilic block copolymer comprises a poloxamine. A poloxamine comprises a central ethylenediamine residue with four -AB and/or -BA side chains. In such embodiments, the -AB or -BA side chains comprise polyoxypropylene units and polyethylene oxide units. Preferably, the poloxamine comprises an ethylenediamine core with four -BA side chains comprising a hydrophobic polyoxypropylene block that is terminated with a hydrophilic polyethylene oxide block. Examples of a suitable poloxamine include poloxamine T1107, poloxamine T304, poloxamine 901, poloxamine 904, poloxamine, 1301, poloxamine 1307, and poloxamine T150R1.

In some of the embodiments herein, the amphiphilic block copolymer comprises a polyol. A polyol is a polymeric compound comprising multiple hydroxy groups and includes compounds, such as polyvinyl alcohol and hydroxy-terminated polymers, such as polyether polyol and polyester polyol. PLURADOT™ polyols are a quad-block surfactant composed of a block copolymer of trimethylolpropane attached to three blocks of polyoxyethylene can be prepared from a low molecular weight trifunctional alcohol, such as glycerine or trimethylpropane, which is oxyalkylated initially with a blend of propylene and ethylene oxides, but primarily with propylene oxide, to form the hydrophobic block. This is followed by oxyalkylating with a blend of ethylene and propylene oxides, but primarily ethylene oxide, to form the hydrophilic block. This group of copolymers has three chains, one more than the poloxamer and meroxapol series, but one less than the poloxamine polymers.

In any of the embodiments herein, the amphiphilic block copolymer comprises a polyethylenimine. A polyethylenimine includes a poly(2-oxazoline) that has been partially or fully deacetylated. The polyethylenimine can be linear or branched, but preferably is branched.

In any of the embodiments herein, the amphiphilic block copolymer comprises a styrene maleic anhydride. A styrene maleic anhydride has repeat units based on styrene and maleic anhydride in varying ratios. Thus, styrene maleic anhydride can be an alternating or random copolymer.

In any of the embodiments of the inventive methods, the amphiphilic block copolymer comprises poloxamer 108 (P108), poloxamer 124 (P124), poloxamer 188 (P188), poloxamer 237 (P237), poloxamer 238 (P238), poloxamer 288 (P288), poloxamer 338 (P338), poloxamer 407 (P407), or poloxamine T1107. In some preferred embodiments, the amphiphilic block copolymer comprises poloxamer 108 (P108), poloxamer 188 (P188), poloxamer 238 (P238), or poloxamine T1107, which are available from BASF Corp. (Parsippany, NJ). Other suitable amphiphilic block copolymers include those under the tradenames LUTROL™, KOLLOPHOR™, PLURONIC™, TETRONIC™, PLURADOT™ and PLURONIC™, which are products available from BASF Corp. (Parsippany, NJ).

The amphiphilic block copolymer can be provided in any suitable manner. For example, the amphiphilic block copolymer can be purchased commercially or synthetically prepared using routine procedures known in the art.

In some embodiments, the amphiphilic block copolymer comprises three or more (e.g., 4 or more, 5 or more, 6 or more, 7 or more, etc.) hydrophobic substituents on a hydrophobic block of the copolymer. In particular, the hydrophobic block can be modified to have three or more, such as 3, 4, 5, 6, 7, 8, 9, 10, 11, 12, 13, 14, or 15, hydrophobic substituents. The hydrophobic substituents can be the same or different, but preferably they are the same. The hydrophobicity (π) of a substituent (x) can be measured by the following equation:

πₓ=log Pₓ−log P_{H},

in which πₓ is the hydrophobicity constant of substituent x, Pₓ is the partition coefficient for a copolymer substituted by x, and P_{H} is the partition coefficient for the corresponding unsubstituted copolymer. For positive values of πₓ, then substituent x is considered to be hydrophobic relative to the unsubstituted copolymer. Suitable hydrophobic substituents include, for example, alkyl, cycloalkyl, haloalkyl, halo, and aryl.

As used herein, the term “alkyl” means a straight or branched, saturated aliphatic radical having a chain containing from, for example, from about 1 to about 12 carbon atoms, e.g., from about 1 to about 10 carbon atoms, from about 1 to about 8 carbon atoms. Cₓ alkyl and Cₓ-C_{y} alkyl are typically used where X and Y indicate the number of carbon atoms in the chain (e.g., C₁-C₁₂ alkyl, C₁-C₁₀ alkyl, C₁-C₈ alkyl, C₁-C₆ alkyl, C₁-C₄ alkyl, C₂-C₆ alkyl, C₂-C₄ alkyl). For example, C₁-C₆ alkyl includes alkyls that have a chain of between 1 and 6 carbons (e.g., methyl, ethyl, propyl, isopropyl, butyl, sec-butyl, isobutyl, tert-butyl, pentyl, neopentyl, isopentyl, n-hexyl, and the like).

The term “cycloalkyl” refers to saturated and partially unsaturated cyclic hydrocarbon groups having 3 to 10 carbons, for example, 3 to 8 carbons, 3 to 6 carbons, or 5 to 6 carbons. C₃-C₁₀ cycloalkyl groups include, for example, cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo [2.2.1]hept-1-yl, and the like.

As used herein, the term “halo” refers to a substituent selected from fluoro, chloro, bromo, and iodo.

A “haloalkyl” refers to an “alkyl” group substituted by one or more “halo” moieties, as such terms are defined in this application. For example, haloalkyl includes haloalkyl, dihaloalkyl, trihaloalkyl, perhaloalkyl, and the like (e.g., halosubstituted (C₁-C₃) alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. In some embodiments, the aryl is phenyl.

In an embodiment, the amphiphilic block copolymer is a poloxamer with an A-B-A structure of formula (I), in which the polyoxypropylene core is modified to have three or more hydrophobic substituents or an alkylene spacer added to the middle of polyoxypropylene core. In particular, the amphiphilic block copolymer is of formula (I), in which the polyoxypropylene core comprises three or more alkyl substituents (e.g., three ethyl substituents) or an alkylene spacer in the middle of the polyoxypropylene repeat units to provide added flexibility and/or better hydrophobic interaction.

In an embodiment, the amphiphilic block copolymer is a poloxamer with an A-B-A structure of formula (I), in which the polyoxypropylene core (B) is modified to have three or more hydrophobic substituents. In particular, the amphiphilic block copolymer is of formula (I), in which the polyoxypropylene core comprises three or more alkyl substituents (e.g., three ethyl substituents).

In an embodiment, the amphiphilic block copolymer is a poloxamer with an A-B-A structure comprising an alkylene spacer added to the middle of the polyoxypropylene core (B). Suitable poloxamers that can include an alkylene spacer are described herein. In particular, the modified poloxamer has a structure of formula (Ia)
(Ia),
   in which each x is an integer of 2 to 130, each y is an integer of 7 to 33, and n is an integer of 1 to 20 (e.g., 1, 2, 3, 4, 5, 6, 7, 8, 9, 10, 11, 12, 13, 14, 15, 16, 17, 18, 19, or 20). In some embodiments, the subscript “n” ranges from 1 to 15, 1 to 10, 3 to 10, 3 to 8, 3 to 6, or 3 to 5 or alternatively, n is 3. The subscript “x” ranges from 2 to 130 (e.g., 9 to 100, 10 to 90, 20 to 80, 40 to 125, 46 to 123, 60 to 100, 62 to 97, 60 to 80, or 62 to 80). Preferably, x is an integer selected from 9, 46, 62, 80, 97, and 122. The subscript “y” ranges from 7 to 33 (e.g., 8 to 23, 8 to 19, 13 to 33, 13 to 23, or 13 to 19). Preferably, y is an integer selected from 8, 13, 19, 23, and 33. In certain embodiments, x is 80 and y is 13 (poloxamer 188) or x is 46 and y is 8 (poloxamer 108) or x is 97 and y is 19 (poloxamer 238) or x is 62 and y is 19 (poloxamer 237) or x is 122 and y is 23 (poloxamer 288).

In a preferred embodiment, the copolymer of formula (Ia) is a species in which x is 80, y is 13, and n is 3:

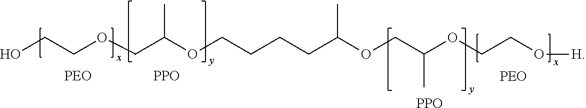

In an embodiment, the invention provides an amphiphilic block copolymer comprising at least one (e.g., 1, 2, 3, 4, 5, etc.) hydrophobic block and at least one (e.g., 1, 2, 3, 4, 5, etc.) hydrophilic block, as described herein, in which the at least one hydrophobic block comprises three or more hydrophobic substituents or an alkylene spacer, as described herein. Without wishing to be bound by any theory, the modified hydrophobic block facilitates hydrophobic domain matching and provides opportunities for enhanced hydrogen bonding and stronger interactions between the copolymer and virus.

The amphiphilic block copolymer can have any suitable number average molecular weight that is suitable for treating a subject, cell, and/or tissue. In general, molecular weight will be selected to provide the appropriate solubility of the amphiphilic block copolymer in water while minimizing or eliminating any potential toxicity. In any of the amphiphilic block copolymers, as the percent of the hydrophilic block increases, or the molecular weight of the hydrophobic block decreases, the solubility of the amphiphilic block copolymer in water increases.

In some embodiments, the length of the hydrophobic block amphiphilic block copolymer can be tailored to match the length of the exposed hydrophobic domain of the target virus.

In some embodiments, the amphiphilic block copolymer has a total number average molecular weight of about 1000 g/mol or more (e.g., 2000 g/mol or more, 3000 g/mol or more, 4000 g/mol or more, 5000 g/mol or more, 6000 g/mol or more, 7000 g/mol or more, 8000 g/mol or more, 9000 g/mol or more, 10,000 g/mol or more, 12,000 g/mol or more, 15,000 g/mol or more, 18,000 g/mol or more, 20,000 g/mol or more, 22,000 g/mol or more, 25,000 g/mol or more, or 28,000 g/mol or more). Alternatively, or in addition, the amphiphilic block copolymer has a total number average molecular weight of about 30,000 g/mol or less (e.g., 28,000 g/mol or less, 25,000 g/mol or less, 22,000 g/mol or less, 20,000 g/mol or less, 18,000 g/mol or less, 15,000 g/mol or less, 12,000 g/mol or less, 10,000 g/mol or less, 9,000 g/mol or less, 8,000 g/mol or less, 7,000 g/mol or less, 6,000 g/mol or less, 5,000 g/mol or less, 4,000 g/mol or less, 3,000 g/mol or less, or 2,000 g/mol or less). Any two of the foregoing endpoints can be used to define a close-ended range, or a single endpoint can be used to define an open-ended range. For example, the amphiphilic block copolymer can have a number average molecular weight within the range of about 1000 to about 30,000 g/mol (e.g., about 3,000 g/mol to about 20,000 g/mol or about 4,000 g/mol to about 18,000 g/mol).

In some embodiments, the molecular weight of the hydrophobic block will make up about 5-55% by weight of the total copolymer, and the molecular weight of the hydrophilic block(s) will make up about 45-95% by weight of the total copolymer. For example, the molecular weight of the hydrophobic block will be about 550 to 16,500 g/mol, and the remainder of the molecular weight can be attributed to the hydrophobic block(s).

The phrase “large molecular weight,” as used herein refers to an amphiphilic block copolymer with a molecular weight of about 8,000 g/mol or more. The phrase “small molecular weight,” as used herein refers to an amphiphilic block copolymer with a molecular weight of less than about 8,000 g/mol.

The number average molecular weight can be measured by any suitable method, including gel permeation chromatography (GPC) and size exclusion chromatography (SEC). Preferably, the number average molecular weight is measured by GPC.

In some embodiments of the methods described herein, the method further comprises administering to the subject a therapeutically effective amount of an antioxidant. It has been found that the presence of an antioxidant with the amphiphilic block copolymer (e.g., poloxamer) stabilizes the block copolymer and helps to prevent its degradation. The antioxidant is any suitable antioxidant and can be, for example, ascorbic acid, ascorbate, tocopherol, retinol, mannitol, a flavonoid (e.g., a bioflavonoid), proanthocyanidin, selenium, gluthathione, N-acetyl-cysteine, superoxide dismutase, lipoic acid, coenzyme Q-10, beta-carotene, lycopene, lutein, polyphenol, or a combination thereof.

A flavonoid is a polyphenol plant metabolite that is soluble in water and has antioxidant properties. Suitable flavonoids include, for example, a chalcone (e.g., isobavachalcone, xanthoangelol, 4-hydroxy-derricin 2′-hydroxy-3,4,5,3′,4′-pentamethoxychalcone and 2′-hydroxy-3,4,5-trimethoxychalcone), an isoflavonoid (e.g., an isoflavone, an isoflavonone, an isoflavan, a pterocarpan, and a rotenoid), a flavonol (e.g., quercetin, kaempferol, myricetin, and fisetin), a flavan-3-ol (e.g., catechin, epicatechin gallate, gallocatechin, and theaflavin), a flavone (apigenin and luteolin), a flavonone (e.g., hesperetin, naringenin, and eriodictyol), an anthocyanidin (e.g., cyanidin, delphinidin, malvidin, pelargonidin, peonidin, and petunidin), an anthoxanthin (e.g., quercetin), and an anthocyanin (e.g., cyanidin, delphinidin, malvidin, pelargonidin, peonidin, and petunidin).

The disease caused by the virus can be, for example, coronavirus disease (COVID-19), severe acute respiratory syndrome (SARS) virus, Middle East respiratory syndrome (MERS), a respiratory disease (e.g., pneumonia, bronchitis, pleural effusion), an inflammatory disease (e.g., inflammation, COVID-19-induced inflammation, pediatric multi-system inflammatory syndrome (PMIS)), reproductive and respiratory syndrome virus (PRRSV), equine arteritis virus (EAV), or gastroenteritis.

The methods of inhibiting an unfolded protein response (UPR) of a virus comprising an exposed hydrophobic domain, preventing death of tissue infected by a virus, and promoting cell repair and recovery to increase survival of cells infected by a virus can each be an in vivo treatment in a subject in need thereof, as described herein, or an in vitro or ex vivo treatment of a cell and/or tissue. The cell can be from any suitable tissue, such as tissue of the respiratory system, including tissue from the lung, nasal cavity, oral cavity, pharynx, trachea, or a combination thereof.

The methods described herein comprise using (e.g., administering) the amphiphilic block copolymer in the form of a pharmaceutical composition. In particular, a pharmaceutical composition will comprise at least one amphiphilic block copolymer, as described herein, and a pharmaceutically acceptable carrier. The pharmaceutically acceptable excipients described herein, for example, vehicles, adjuvants, carriers or diluents, are well-known to those who are skilled in the art and are readily available to the public. Typically, the pharmaceutically acceptable carrier is one that is chemically inert to the amphiphilic block copolymer and one that has no detrimental side effects or toxicity under the conditions of use.

The amphiphilic block copolymer or pharmaceutical composition can be administered as oral, sublingual, transdermal, subcutaneous, topical, absorption through epithelial or mucocutaneous linings, intravenous, intranasal, intraarterial, intramuscular, interperitoneal, intrathecal, rectal, vaginal, or aerosol formulations. In some aspects, the amphiphilic block copolymer or pharmaceutical composition is administered intravenously, subcutaneously, or topically.

In accordance with any of the embodiments, the amphiphilic block copolymer can be administered orally to a subject in need thereof. Formulations suitable for oral administration can consist of (a) liquid solutions, such as an effective amount of the amphiphilic block copolymer dissolved in diluents, such as water (e.g., sterile and/or distilled water), saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The amphiphilic block copolymer can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as polyethylene glycol (e.g., PEG400), an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the amphiphilic block copolymer in solution. Suitable preservatives and buffers can be used in such formulations. Amphiphilic copolymers can be administered as preparations that are substantially reduced in polydispersity (i.e., purified) to remove components less than 2,500 g/mol that have or give rise to a longer tissue or blood half-life of the copolymer. A shorter half-life leads to more rapid achievement of tissue therapeutic levels. Preferably, amphiphilic copolymers that are less than 3,500 Da in size, with half-lives that are 2-fold or greater more than the main active agent are removed from the composition.

The amphiphilic block copolymer may be made into an injectable formulation. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topically applied compositions are generally in the form of liquids (e.g., mouthwash), creams, pastes, lotions and gels. Topical administration includes application to the oral mucosa, which includes the oral cavity, oral epithelium, palate, gingival, and the nasal mucosa. In some embodiments, the composition contains at least one amphiphilic block copolymer and a suitable vehicle or carrier. It may also contain other components, such as an anti-irritant. The carrier can be a liquid, solid or semi-solid. In embodiments, the composition is an aqueous solution, such as a mouthwash. Alternatively, the composition can be a dispersion, emulsion, gel, lotion or cream vehicle for the various components. In one embodiment, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In embodiments of the invention, the vehicle for topical application to the skin can include water, buffered solutions, various alcohols, glycols such as glycerin, lipid materials such as fatty acids, mineral oils, phosphoglycerides, collagen, gelatin, and silicone-based materials.

The amphiphilic block copolymer, alone or in combination with other suitable components, can be made into aerosol formulations to be administered via inhalation. These aerosol formulations can be placed into pressurized acceptable propellants. Suitable propellants include, e.g., a fluorinated hydrocarbon (e.g., trichloromonofluoromethane, dichlorodifluoromethane, chlorodifluoromethane, chlorodifluoroethane, dichlorotetrafluoroethane, heptafluoropropane, tetrafluoroethane, difluoroethane), a hydrocarbon (e.g., propane, butane, isobutane), or a compressed gas (e.g., nitrogen, nitrous oxide, carbon dioxide). The amphiphilic block copolymer may also be formulated as pharmaceuticals for non-pressured preparations, such as in a nebulizer or an atomizer.

The dose administered to the subject, particularly human and other mammals, in accordance with the present invention should be sufficient to affect the desired response. One skilled in the art will recognize that dosage will depend upon a variety of factors, including the age, condition or disease state, predisposition to disease, genetic defect or defects, and body weight of the subject. The size of the dose will also be determined by the route, timing and frequency of administration as well as the existence, nature, and extent of any adverse side-effects that might accompany the administration of a particular amphiphilic block copolymer and the desired effect. It will be appreciated by one of ordinary skill in the art that various conditions or disease states may require prolonged treatment involving multiple administrations.

The inventive methods comprise using an effective amount of the amphiphilic block copolymer. An “effective amount” means an amount sufficient to show a meaningful benefit in an individual, cell, or tissue to be treated. A meaningful benefit includes, for example, detectably treating, relieving, or lessening one or more symptoms of a disease caused by a virus (e.g., inflammation, fluid accumulation), inhibiting, arresting development, preventing, or halting further development of the viral infection or disease, reducing the incidence of a disease caused by virus, preventing death of tissue infected by a virus, promoting cell repair and recovery to increase survival of cells infected by a virus, inhibiting a gene transcription response, inhibiting a cellular metabolic response, and/or detectably inhibit virus replication and/or inhibit an unfolded protein response of a virus comprising an exposed hydrophobic domain in a subject, cell, or tissue. The meaningful benefit observed in the subject, cell, or tissue to be treated can be to any suitable degree (10, 20, 30, 40, 50, 60, 70, 80, 90% or more). In some aspects, one or more symptoms of the disease are prevented, reduced, halted, or eliminated subsequent to administration of an amphiphilic block copolymer described herein, thereby effectively treating the disease to at least some degree.

Effective amounts may vary depending upon the biological effect desired in the individual, cell and/or tissue to be treated, condition to be treated, and/or the specific characteristics of the amphiphilic block copolymer. In this respect, any suitable dose of the amphiphilic block copolymer can be administered to the subject (e.g., human), cell, or tissue. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the amphiphilic block copolymer desirably comprises about 0.00001 mg per kilogram (kg) of the body weight of the subject or more (e.g., about 0.00005 mg/kg or more, 0.0001 mg/kg or more, 0.0005 mg/kg or more, 0.001 mg/kg or more, 0.005 mg/kg or more, 0.01 mg/kg or more, 0.05 mg/kg or more, 0.1 mg/kg or more, 0.5 mg/kg or more, 1 mg/kg or more, 2 mg/kg or more, 5 mg/kg or more, 10 mg/kg or more, 15 mg/kg or more, 20 mg/kg or more, 30 mg/kg or more, 40 mg/kg or more, 50 mg/kg or more, 75 mg/kg or more, 100 mg/kg or more, 125 mg/kg or more, 150 mg/kg or more, 175 mg/kg or more, 200 mg/kg or more, 225 mg/kg or more, 250 mg/kg or more, 275 mg/kg or more, 300 mg/kg or more, 325 mg/kg or more, 350 mg/kg or more, 375 mg/kg or more, 400 mg/kg or more, 425 mg/kg or more, 450 mg/kg or more, or 475 mg/kg or more) per day. Typically, the dose will be about 500 mg/kg or less (e.g., about 475 mg/kg or less, about 450 mg/kg or less, about 425 mg/kg or less, about 400 mg/kg or less, about 375 mg/kg or less, about 350 mg/kg or less, about 325 mg/kg or less, about 300 mg/kg or less, about 275 mg/kg or less, about 250 mg/kg or less, about 225 mg/kg or less, about 200 mg/kg or less, about 175 mg/kg or less, about 150 mg/kg or less, about 125 mg/kg or less, about 100 mg/kg or less, about 75 mg/kg or less, about 50 mg/kg or less, about 40 mg/kg or less, about 30 mg/kg or less, about 20 mg/kg or less, about 15 mg/kg or less, about 10 mg/kg or less, about 5 mg/kg or less, about 2 mg/kg or less, about 1 mg/kg or less, about 0.5 mg/kg or less, or about 0.1 mg/kg or less). Any two of the foregoing endpoints can be used to define a close-ended range, or a single endpoint can be used to define an open-ended range.

For purposes of the present invention, the term “subject” preferably is directed to a mammal. Mammals include, but are not limited to, the order Rodentia, such as mice, and the order Lagomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perissodactyla, including Equines (horses). It is most preferred that the mammals are of the order Primates, Cebids, or Simioids (monkeys) or of the order Anthropoids (humans and apes). An especially preferred mammal is a human.

A subject in need thereof is any one that has come in contact with, suspected to have come in contact with, or expected to come into contact with a virus, particularly a virus comprising an exposed hydrophobic domain (e.g., SARS-CoV-2). At risk subjects for developing a disease caused by a virus that include, for example, people aged 40 and older (particularly people aged 60 and older), unvaccinated people, people with one more underlying conditions (e.g., cardiovascular disease, Down syndrome, sickle cell disease, diabetes (type 1 or type 2), chronic respiratory disease (including chronic obstructive pulmonary disease, interstitial lung disease, cystic fibrosis), asthma, dementia, Alzheimer's disease, liver disease chronic kidney disease undergoing dialysis, high blood pressure, obesity (e.g., a body mass index (BMI) of 30 or higher, especially 40 or higher), and cancer), people that are immunocompromised (e.g., due to a condition, such as smoking, substance use disorder, cancer treatment, bone marrow or organ transplantation, HIV, AIDs, and prolonged use of corticosteroids and other immune weakening treatments), and people living in a nursing home or a long-term care facility.

The following are certain aspects of the invention.

1. A method of treating a disease caused by a virus in a subject in need thereof comprising administering to the subject a therapeutically effective amount of an amphiphilic block copolymer.

2. A method of inhibiting virus replication in a subject infected by a virus comprising administering to the subject a therapeutically effective amount of an amphiphilic block copolymer.

3. A method of inhibiting an unfolded protein response of a virus comprising an exposed hydrophobic domain comprising contacting the exposed hydrophobic domain with an amphiphilic block copolymer.

4. A method of preventing death of tissue infected by a virus comprising contacting the infected tissue with an amphiphilic block copolymer.

5. A method of promoting cell repair and recovery to increase survival of cells infected by a virus comprising contacting the infected cells with an amphiphilic block copolymer.

6. The method of any one of aspects 1-5, wherein the virus is an RNA virus selected from a coronavirus, a flavivirus, a rhabdovirus, an orthmyxovirus, a hepevirus, a herpesvirus, and a retrovirus.

7. The method of aspect 6, wherein the RNA virus is a coronavirus.

8. The method of any one of aspects 1-5, wherein the virus is a DNA virus selected from a hepadnavirus, an asfarvirus, a papillomavirus, and a poxvirus.

9. The method of any one of aspects 1-8, wherein the amphiphilic block copolymer comprises at least one hydrophobic block comprising repeat units selected from a hydrophobic polypeptide, polyoxypropylene, polystyrene, polyglycolide, polylactide, poly(lactic-glycoacid), polycaprolactone, hydrophobic polyurethane, polyester, poly-N-isopropylacrylamide, polymethylmethacrylate, poly(2-dimethylamino ethylmethacrylate), polyethylene, polypropylene, polyisoprene, polybutylene, polybutadiene, poly(styrene-butadiene), polyvinyl chloride, polytetrafluoroethylene, polydimethylsiloxane, and a combination thereof, and at least one hydrophilic block comprising repeat units selected from polyethylene oxide, polyvinyl alcohol, hydrophilic polyurethane, polyvinylpyrrolidone, polyacrylamide, polyacrylic acid, poly(meth)acrylic acid, polyethylenimine, poly(methyl vinyl ether), poly(styrene-maleic anhydride), polyethylene glycol ether, polyamine, a hydrophilic polypeptide, and a combination thereof.

10. The method of any one of aspects 1-9, wherein the amphiphilic block copolymer comprises a polypeptide, a poloxamer, a meroxapol, a poloxamine, a polyol, a polyethylenimine, a styrene maleic anhydride, or a combination thereof.

11. The method of aspect 10, wherein the amphiphilic block copolymer comprises a polyol of trimethylolpropane and polyoxyethylene.

12. The method of any one of aspects 1-11, wherein the amphiphilic block copolymer comprises poloxamer 108, poloxamer 188, poloxamer 238, or poloxamine T1107.

13. The method of any one of aspects 1-12, wherein the amphiphilic block copolymer has a number average molecular weight of about 1000 to about 30,000 g/mol.

14. The method of any one of aspects 1-7 and 9-13, wherein the disease is coronavirus disease (COVID-19), SARS virus, MERS, a respiratory disease, or an inflammatory disease.

15. The method of any one of aspects 1, 2, and 6-14, wherein the amphiphilic block copolymer is administered intravenously, subcutaneously, or topically to the subject.

16. The method of any one of aspects 1, 2, and 6-15, wherein the method further comprises administering to the subject a therapeutically effective amount of an antioxidant.

17. The method of aspect 16, wherein the antioxidant is selected from ascorbic acid, ascorbate, tocopherol, retinol, mannitol, a flavonoid, proanthocyanidin, selenium, gluthathione, N-acetyl-cysteine, superoxide dismutase, lipoic acid, coenzyme Q-10, beta-carotene, lycopene, lutein, polyphenol, and a combination thereof.

18. The method of any one of aspects 1-17, wherein the amphiphilic block copolymer comprises three or more hydrophobic substituents on a hydrophobic block of the copolymer that are the same or different and each is selected from alkyl, cycloalkyl, haloalkyl, halo, and aryl.

19. The method of aspect 18, wherein the amphiphilic block copolymer comprising a hydrophobic polyoxypropylene core and two hydrophilic polyethylene oxide side chains of formula (I):

HO—(C₂H₄O)_{b}—(C₃H₆O)ₐ—(C₂H₄O)_{b}—H (I),

wherein a is an integer such that the hydrophobic polyoxypropylene core has a molecular weight of about 500-15,500 g/mol, and b is an integer such that the hydrophilic polyethylene oxide side chains constitute about 50-90% by weight of the copolymer.

20. The method of aspect 18 or 19, wherein the hydrophobic polyoxypropylene core comprises three or more alkyl substituents, preferably three ethyl substituents.

21. An amphiphilic block copolymer comprising three or more hydrophobic substituents on a hydrophobic block of the copolymer.

22. The amphiphilic block copolymer of aspect 21, wherein the three or more hydrophobic substituents are the same or different and each is selected from alkyl, cycloalkyl, haloalkyl, halo, and aryl.

23. The amphiphilic block copolymer of aspect 21 or 22, wherein the amphiphilic block copolymer comprising a hydrophobic polyoxypropylene core and two hydrophilic polyethylene oxide side chains of formula (I):

HO—(C₂H₄O)_{b}—(C₃H₆O)ₐ—(C₂H₄O)_{b}—H (I),

wherein a is an integer such that the hydrophobic polyoxypropylene core has a molecular weight of about 500-15,500 g/mol, and b is an integer such that the hydrophilic polyethylene oxide side chains constitute about 50-90% by weight of the copolymer.

24. The amphiphilic block copolymer of aspect 21, wherein the copolymer comprises an alkylene spacer and has a structure of formula (Ia):
(Ia),
   wherein each x is an integer of 2 to 130, each y is an integer of 7 to 33, and n is an integer of 1 to 20.

It shall be noted that the preceding are merely examples of embodiments. Other exemplary embodiments are apparent from the entirety of the description herein. It will also be understood by one of ordinary skill in the art that each of these embodiments may be used in various combinations with the other embodiments provided herein.

The following examples further illustrate the invention but, of course, should not be construed as in any way limiting its scope.

### Example 1

This example demonstrates the ability of an amphiphilic block copolymer to reduce infection of a virus.

OC43 is a human beta coronavirus that is responsible for the common cold. The screening involves testing human lung cell response to the OC43 CoV. Cells were contacted with a solution of 3 mM the amphiphilic block copolymer comprises poloxamer 108 (P108) (4700 g/mol), poloxamer 188 (P188) (8400 g/mol), poloxamer 238 (P238) (11,400 g/mol), or poloxamine T1107 (15,000 g/mol), which are available from BASF Corp. (Parsippany, NJ). The high concentration of copolymer was required because the cell treatment was performed without media convection. In the proposed embodiment, such as in the body, the required concentration would be substantially lower.

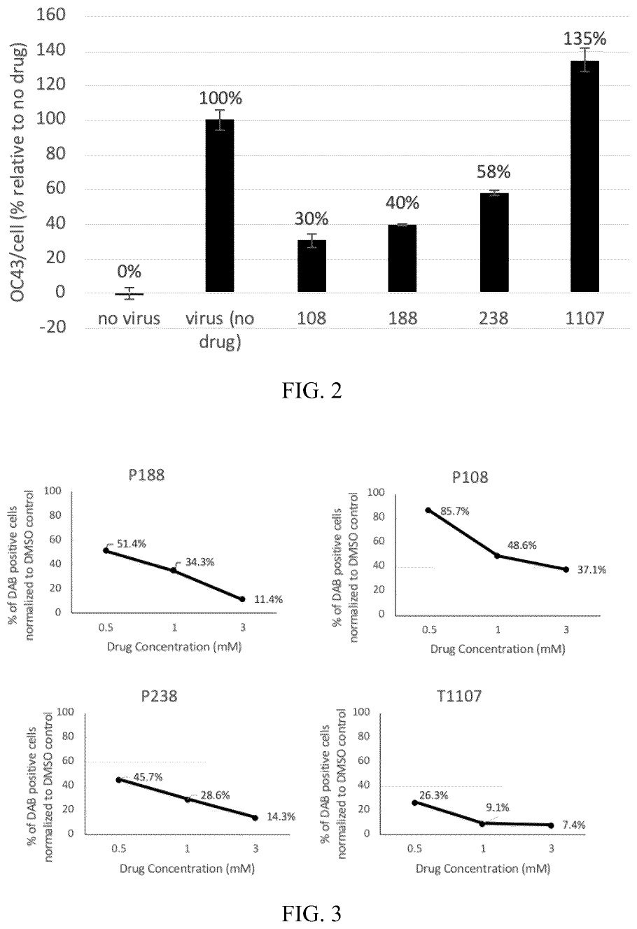

The cells were tested for infection 4 days post-exposure. The fluorescence per cell was calculated and normalized to infected cells with no amphiphilic block copolymer (control). The results are shown in FIG. 2.

Poloxamers of lower molecular weight had greater efficacy in reducing OC43 infection.

### Example 2

This example demonstrates the ability of an amphiphilic block copolymer to reduce infection of a virus.

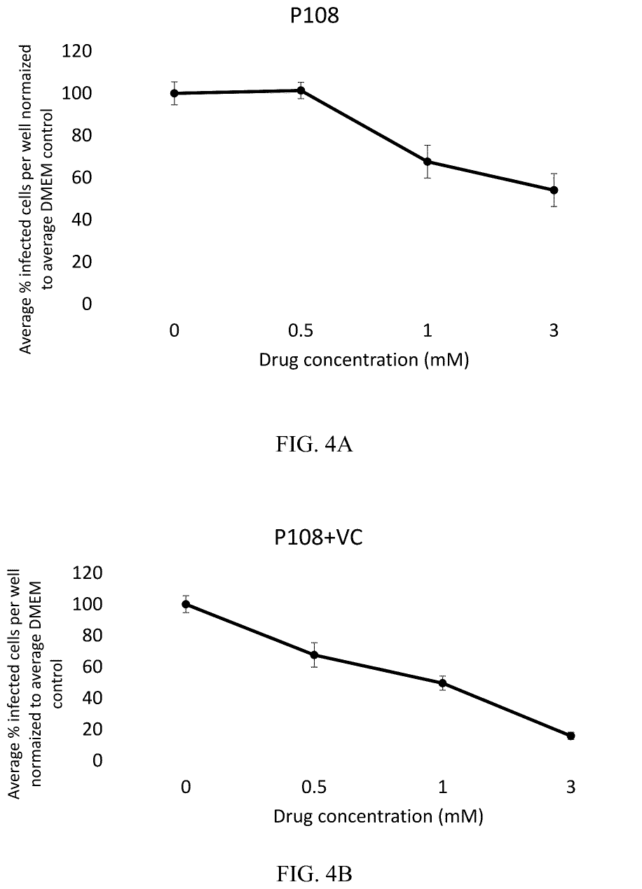

Example 1 was replicated using SARS-CoV-2 virus. Cells were contacted with 0.5 mM, 1 mM, and 3 mM of poloxamer 108 (P108) (4700 g/mol), poloxamer 188 (P188) (8400 g/mol), poloxamer 238 (P238) (11,400 g/mol), or poloxamine T1107 (15,000 g/mol). The amount of SARS-CoV-2 per cell was calculated and normalized to infected cells with no amphiphilic block copolymer (control). The resulting dose response curves are shown in FIG. 3.

Poloxamers of larger sizes were able to reduce SARS-CoV-2 infection more effectively. The mechanism of coronavirus entry into the cell is different for the OC43 strain than it is for the SARS-CoV-2. Not bound by any theory, this difference may explain why the molecular weight range of effective poloxamers is larger for SARS-CoV-2 than for OC43.

### Example 3

This example demonstrates the ability of an amphiphilic block copolymer in combination with an antioxidant to reduce infection of a virus.

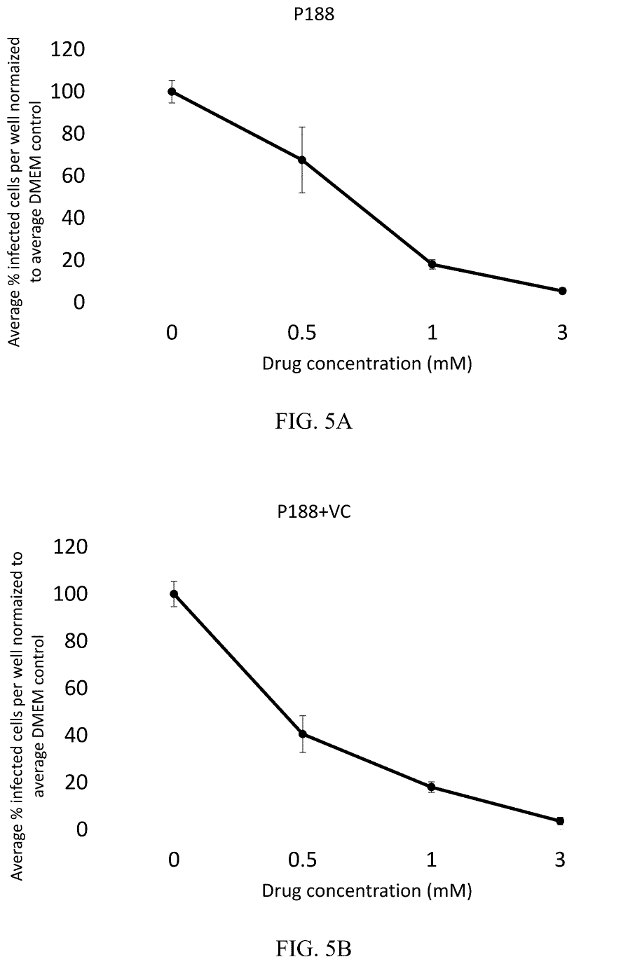

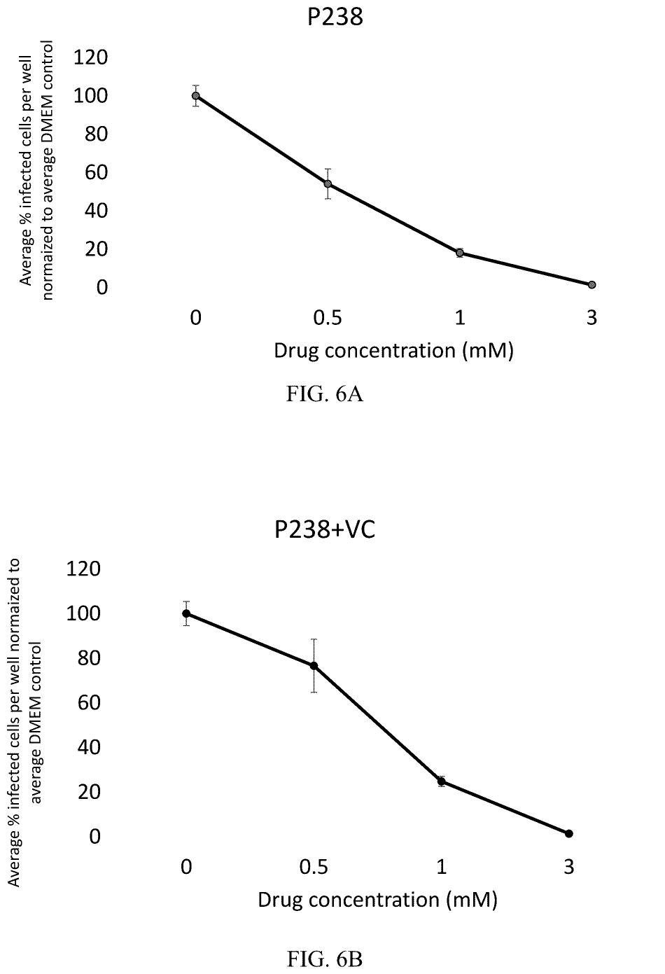

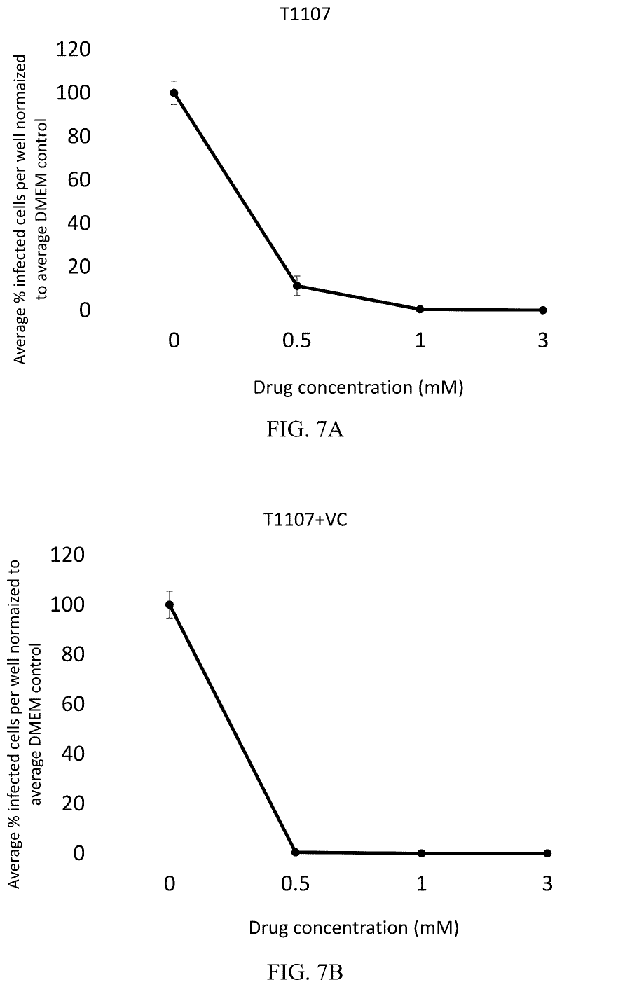

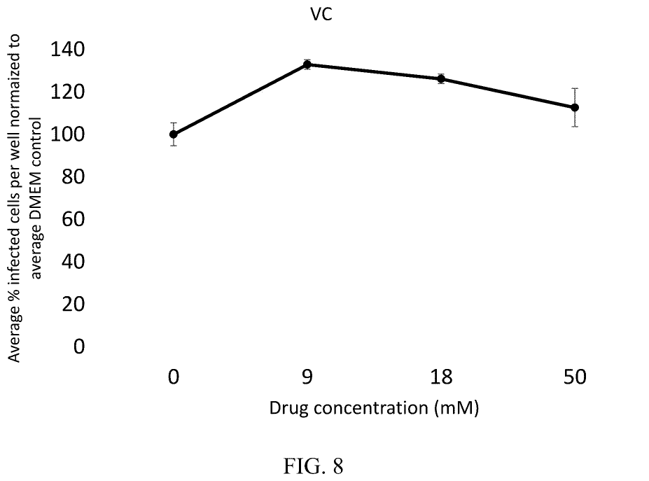

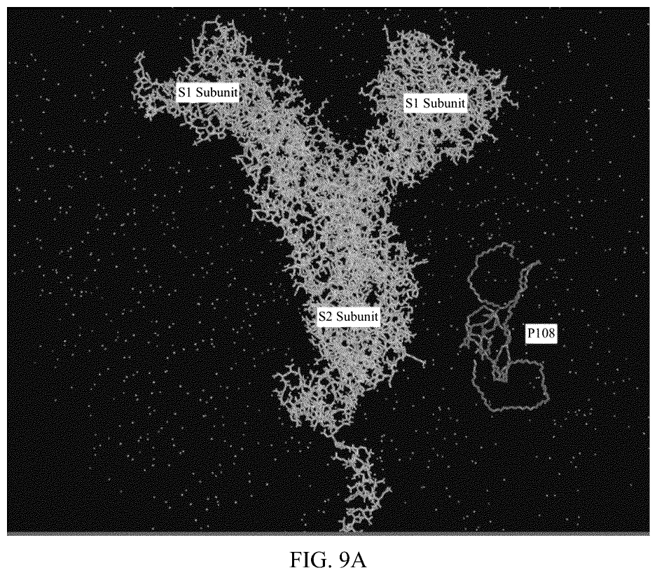

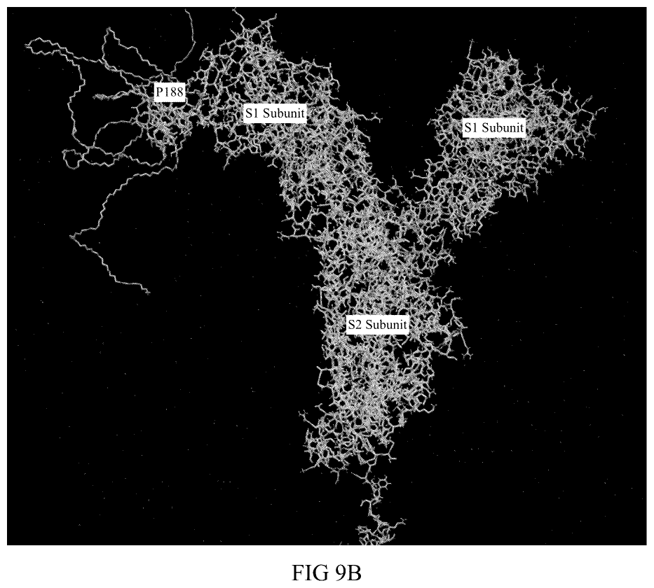

The test of Example 1 was replicated using SARS-CoV-2. Cells were contacted with 0 mM (control), 0.5 mM, 1 mM, and 3 mM of either poloxamer 108 (P108) (4700 g/mol), poloxamer 188 (P188) (8400 g/mol), poloxamer 238 (P238) (11,400 g/mol), or poloxamine T1107 (15,000 g/mol)—each with or without ascorbate (Vitamin C, “VC”) at 50 μM. The amount of SARS-CoV-2 per cell was calculated and normalized to infected cells with no amphiphilic block copolymer or VC (control). The resulting dose response curves are shown in FIGS. 4-7. As another control, VC was administered in the absence of an amphiphilic block copolymer in doses of 0 mM (control), 9 mM, 18 mM, and 50 mM. The resulting dose response curves are shown in FIG. 8.

As seen in FIGS. 4-8, the presence of an antioxidant surprisingly improved the ability of the amphiphilic block copolymers to reduce the viral infection content.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A method of treating a disease caused by a virus in a subject in need thereof comprising administering to the subject a therapeutically effective amount of an amphiphilic block copolymer.

2. The method of claim 1, wherein the virus is an RNA virus selected from a coronavirus, a flavivirus, a rhabdovirus, an orthmyxovirus, a hepevirus, a herpesvirus, and a retrovirus.

3. The method of claim 2, wherein the RNA virus is a coronavirus.

4. The method of claim 1, wherein the disease is coronavirus disease (COVID-19), SARS virus, MERS, a respiratory disease, or an inflammatory disease.

5. The method of claim 1, wherein the amphiphilic block copolymer comprises
at least one hydrophobic block comprising repeat units selected from a hydrophobic polypeptide, polyoxypropylene, polystyrene, polyglycolide, polylactide, poly(lactic-glycoacid), polycaprolactone, hydrophobic polyurethane, polyester, poly-N-isopropylacrylamide, polymethylmethacrylate, poly(2-dimethylamino ethylmethacrylate), polyethylene, polypropylene, polyisoprene, polybutylene, polybutadiene, poly(styrene-butadiene), polyvinyl chloride, polytetrafluoroethylene, polydimethylsiloxane, and a combination thereof, and
at least one hydrophilic block comprising repeat units selected from polyethylene oxide, polyvinyl alcohol, hydrophilic polyurethane, polyvinylpyrrolidone, polyacrylamide, polyacrylic acid, poly(meth)acrylic acid, polyethylenimine, poly(methyl vinyl ether), poly(styrene-maleic anhydride), polyethylene glycol ether, polyamine, a hydrophilic polypeptide, and a combination thereof.

6. The method of claim 5, wherein the amphiphilic block copolymer comprises three or more hydrophobic substituents on the hydrophobic block of the copolymer that are the same or different and each is selected from alkyl, cycloalkyl, haloalkyl, halo, and aryl.

7. The method of claim 1, wherein the amphiphilic block copolymer comprises a polypeptide, a poloxamer, a meroxapol, a poloxamine, a polyol, a polyethylenimine, a styrene maleic anhydride, or a combination thereof.

8. The method of claim 7, wherein the amphiphilic block copolymer comprises a polyol of trimethylolpropane and polyoxyethylene.

9. The method of claim 8, wherein the amphiphilic block copolymer comprises poloxamer 108, poloxamer 188, poloxamer 238, or poloxamine T1107.

10. The method of claim 1, wherein the amphiphilic block copolymer has a number average molecular weight of about 1000 to about 30,000 g/mol.

11. The method of claim 1, wherein the method further comprises administering to the subject a therapeutically effective amount of an antioxidant.

12. The method of claim 11, wherein the antioxidant is selected from ascorbic acid, ascorbate, tocopherol, retinol, mannitol, a flavonoid, proanthocyanidin, selenium, gluthathione, N-acetyl-cysteine, superoxide dismutase, lipoic acid, coenzyme Q-10, beta-carotene, lycopene, lutein, polyphenol, and a combination thereof.

13. A method of inhibiting virus replication in a subject infected by a virus comprising administering to the subject a therapeutically effective amount of an amphiphilic block copolymer.

14. The method of claim 13, wherein the amphiphilic block copolymer comprises
at least one hydrophobic block comprising repeat units selected from a hydrophobic polypeptide, polyoxypropylene, polystyrene, polyglycolide, polylactide, poly(lactic-glycoacid), polycaprolactone, hydrophobic polyurethane, polyester, poly-N-isopropylacrylamide, polymethylmethacrylate, poly(2-dimethylamino ethylmethacrylate), polyethylene, polypropylene, polyisoprene, polybutylene, polybutadiene, poly(styrene-butadiene), polyvinyl chloride, polytetrafluoroethylene, polydimethylsiloxane, and a combination thereof, and
at least one hydrophilic block comprising repeat units selected from polyethylene oxide, polyvinyl alcohol, hydrophilic polyurethane, polyvinylpyrrolidone, polyacrylamide, polyacrylic acid, poly(meth)acrylic acid, polyethylenimine, poly(methyl vinyl ether), poly(styrene-maleic anhydride), polyethylene glycol ether, polyamine, a hydrophilic polypeptide, and a combination thereof.

15. A method of inhibiting a cellular metabolic response, a gene transcription response, or an unfolded protein response of a virus comprising an exposed hydrophobic domain comprising contacting the exposed hydrophobic domain with an amphiphilic block copolymer.

16. The method of claim 15, wherein the amphiphilic block copolymer comprises
at least one hydrophobic block comprising repeat units selected from a hydrophobic polypeptide, polyoxypropylene, polystyrene, polyglycolide, polylactide, poly(lactic-glycoacid), polycaprolactone, hydrophobic polyurethane, polyester, poly-N-isopropylacrylamide, polymethylmethacrylate, poly(2-dimethylamino ethylmethacrylate), polyethylene, polypropylene, polyisoprene, polybutylene, polybutadiene, poly(styrene-butadiene), polyvinyl chloride, polytetrafluoroethylene, polydimethylsiloxane, and a combination thereof, and
at least one hydrophilic block comprising repeat units selected from polyethylene oxide, polyvinyl alcohol, hydrophilic polyurethane, polyvinylpyrrolidone, polyacrylamide, polyacrylic acid, poly(meth)acrylic acid, polyethylenimine, poly(methyl vinyl ether), poly(styrene-maleic anhydride), polyethylene glycol ether, polyamine, a hydrophilic polypeptide, and a combination thereof.

17. A method of preventing death of tissue infected by a virus comprising contacting the infected tissue with an amphiphilic block copolymer.

18. The method of claim 17, wherein the amphiphilic block copolymer comprises
at least one hydrophobic block comprising repeat units selected from a hydrophobic polypeptide, polyoxypropylene, polystyrene, polyglycolide, polylactide, poly(lactic-glycoacid), polycaprolactone, hydrophobic polyurethane, polyester, poly-N-isopropylacrylamide, polymethylmethacrylate, poly(2-dimethylamino ethylmethacrylate), polyethylene, polypropylene, polyisoprene, polybutylene, polybutadiene, poly(styrene-butadiene), polyvinyl chloride, polytetrafluoroethylene, polydimethylsiloxane, and a combination thereof, and
at least one hydrophilic block comprising repeat units selected from polyethylene oxide, polyvinyl alcohol, hydrophilic polyurethane, polyvinylpyrrolidone, polyacrylamide, polyacrylic acid, poly(meth)acrylic acid, polyethylenimine, poly(methyl vinyl ether), poly(styrene-maleic anhydride), polyethylene glycol ether, polyamine, a hydrophilic polypeptide, and a combination thereof.

19. A method of promoting cell repair and recovery to increase survival of cells infected by a virus comprising contacting the infected cells with an amphiphilic block copolymer.

20. The method of claim 19, wherein the amphiphilic block copolymer comprises
at least one hydrophobic block comprising repeat units selected from a hydrophobic polypeptide, polyoxypropylene, polystyrene, polyglycolide, polylactide, poly(lactic-glycoacid), polycaprolactone, hydrophobic polyurethane, polyester, poly-N-isopropylacrylamide, polymethylmethacrylate, poly(2-dimethylamino ethylmethacrylate), polyethylene, polypropylene, polyisoprene, polybutylene, polybutadiene, poly(styrene-butadiene), polyvinyl chloride, polytetrafluoroethylene, polydimethylsiloxane, and a combination thereof, and
at least one hydrophilic block comprising repeat units selected from polyethylene oxide, polyvinyl alcohol, hydrophilic polyurethane, polyvinylpyrrolidone, polyacrylamide, polyacrylic acid, poly(meth)acrylic acid, polyethylenimine, poly(methyl vinyl ether), poly(styrene-maleic anhydride), polyethylene glycol ether, polyamine, a hydrophilic polypeptide, and a combination thereof.

21. An amphiphilic block copolymer comprising three or more hydrophobic substituents or an alkylene spacer on a hydrophobic block of the copolymer.

22. The amphiphilic block copolymer of claim 21, wherein the copolymer comprises three or more hydrophobic substituents that are the same or different and each is selected from alkyl, cycloalkyl, haloalkyl, halo, and aryl.

23. The amphiphilic block copolymer of claim 22, wherein the amphiphilic block copolymer comprising a hydrophobic polyoxypropylene core and two hydrophilic polyethylene oxide side chains of formula (I):
HO—(C₂H₄O)_{b}—(C₃H₆O)ₐ—(C₂H₄O)_{b}—H (I),
wherein a is an integer such that the hydrophobic polyoxypropylene core has a molecular weight of about 500-15,500 g/mol, and b is an integer such that the hydrophilic polyethylene oxide side chains constitute about 50-90% by weight of the copolymer.

24. The amphiphilic block copolymer of claim 21, wherein the copolymer comprises an alkylene spacer and has a structure of formula (Ia):
(Ia),
wherein
each x is an integer of 2 to 130,
each y is an integer of 7 to 33, and
n is an integer of 1 to 20.
